(19) United States
(12) Patent Application Publication
(10) Pub. No.: US 2010/0129935 A1
(43) Pub. Date: May 27, 2010

(54) PREGNANCY TESTING METHOD

(76) Inventor: Sarah Daniel Maddison, Raleigh, NC (US)

Correspondence Address: PASSE' INTELLECTUAL PROPERTY, LLC, 1717 BRASSFIELD RD., RALEIGH, NC, 27614, US

(21) Appl. No.: 12/323,083

(22) Filed: Nov. 25, 2008

(51) Int. Cl. G01N 33/53; G01N 31/22

(52) U.S. Cl. 436/510; 422/56

(57) ABSTRACT

The present invention relates to a method of determining if a patient is pregnant and if the pregnancy is progressing. The method involves testing the patient a first time with a hCG home urine test, then after a select number of days, a second hCG home urine test is taken having a lower hCG sensitivity than the first.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a simple home testing method for confirming a viable pregnancy. In particular the present invention relates to testing an individual over two or more periods to determine if the key pregnancy hormone is increasing thus indicating a successful pregnancy. Furthermore, when the hormone is noted not to rise appropriately, the individual can be alerted to the possibility of a miscarriage and/or ectopic (tubal) pregnancy.

2. Description of Related Art

A pregnancy test, especially a home pregnancy type test, is done to determine if a female is pregnant. Normally, the chemical pregnancy test searches for chemical markers found in urine and blood that are associated with pregnancy. Testing (sampling) requires sampling of one of these fluids. Typically, home pregnancy testing relies on urine samples. The urine is poured over a test strip with a color changing chemical marker indicator. A color change can indicate, after a period of time, either a positive or negative pregnancy. The most commonly used marker is hCG, the human chorionic gonadotropin, originally discovered in 1930 to be produced by the placenta during pregnancy and excreted into the urine.

It is known that hCG is produced by trophoblast cells of the placenta in pregnancy. It is also produced in gestational trophoblastic diseases (hydatidiform mole, choriocarcinoma, and placental site trophoblastic tumors) and in testicular germ cell malignancies. The complete hCG protein is a glycoprotein composed of 2 dissimilar subunits, an alpha and beta subunit, held together by charge interactions. The hCG beta subunit is composed of 92 amino acids and contains 2 N-linked oligosaccharides. The hCG alpha subunit is composed of 145 amino acids and contains 2 N-linked and 4 O-linked oligosaccharides. The 8 oligosaccharide side chains comprise greater than 30% of the molecular weight of hCG, making it an exceptionally highly glycosylated glycoprotein. The beta subunit is the primary unit being detected in home pregnancy testing units. The beta hCG rises in a predictable logarithmic pattern in early pregnancy.

Over 40 professional laboratory serum hCG tests, approximately 30 point of care serum and urine hCG tests and a similar number of home pregnancy tests are sold today for detecting hCG and for establishing the onset of pregnancy. Whether intended for professional laboratory or home use, today all pregnancy tests work on the multi-antibody immunometric assay principal: commonly one and occasionally two antibodies (mono- or polyclonal) binds and immobilizes hCG and a second antibody, the tracer antibody, raised to a distant (different) epitope and labeled with an enzyme, dye or chemiluminescence agent, marks the presence of hCG or quantitates hCG present in the sample.

The hCG glycoprotein is a heterogeneous molecule. Cleaved or nicked forms of hCG, free subunits of hCG, and fragments of hCG are all detectable in serum and urine samples during pregnancy. Variable detection or lack of detection of cleaved molecules, free subunits and fragments is a major cause of inter-assay variation in hCG results.

The first home pregnancy testing kits, using an hCG test, were released in the mid-1970's, however, they do create false positives in spite of the fact that hCG is a reliable marker. There are a variety of products on the market that all work slightly different and have a range of sensitivities for hCG from about 20 mIU/mL to 100 mIU/mL, more or less. Problems occur with pregnancy testing in this manner for a variety of reasons including errors of test application, use of drugs containing the assay molecule and non-pregnant production of the chemical. In addition, because the sensitivity of the available home testing units for hCG varies greatly, false negatives can occur if the test is given too early. One particular problem occurs when a home pregnancy test is performed and shows a positive reading, but the individual has already miscarried or spontaneously aborted the pregnancy. The urine will still test positive for hCG, but the individual is unaware that the level has plateaued or is falling which would indicate a potential pregnancy problem. There is currently no simple home method for confirming viable pregnancy.

When pregnancy viability is in question, the status of the pregnancy can be followed with serial quantitative levels that confirm the expected doubling approximately every forty-eight hours. This currently can only be achieved with consecutive blood levels, thus requiring physician and phlebotomy services.

Examples of pregnancy testing are US patent application 2006/0105411 which relates to methods for analyzing concentrations of hyperglycosylated hCG and hCG equally in blood, serum or urine samples, to determine with high accuracy if hCG levels indicate a pregnancy (whether active or recently terminated).In US patent application 2008/0076190 to Carlisle and Tolley published Mar. 27, 2008 there is disclosed a fluidic assay device for assaying at least one property of a liquid in the presence of an analyte and indicating pregnant or not pregnant.

### BRIEF SUMMARY OF THE INVENTION

The present invention relates to a method for testing at home to confirm a pregnancy by taking a first set of urine hCG tests followed at a predetermined time by one or more urine hCG tests of lesser sensitivity. This invention would allow the individual to attain the same assurance of a viable pregnancy through a series of progressively less sensitive qualitative test where the individual would see additional positive indicators only when the hCG was continuing to rise.

In one embodiment, the present invention is a home pregnancy test kit comprising two or more test strips, a first test strip having a first hCG home urine test of a first hCG sensitivity and the second and subsequent tests strips comprising at least one hCG home urine test less sensitive to hCG than the first hCG home urine test.

A second embodiment method of confirming an ongoing pregnancy in an individual comprising the steps of:
a) performing on the individual, a first hCG urine test of a first sensitivity with a first test strip at a given time;
b) performing on the individual at a predetermined time after the first test, a second urine hCG test with a second test strip, the second test strip having at least one hCG urine test having a lesser sensitivity to hCG than the first sensitivity; and
c) determining an ongoing pregnancy confirmation when the first test on the first test strip is positive and the less sensitive test on the second strip is positive.

A third embodiment of the present invention comprises a method of testing in an individual, the progress of a pregnancy comprising testing the individual at, at least 2 predetermined time intervals with a hCG urine test the sensitivity of at least one hCG urine test taken at each interval is successively less sensitive than at least one hCG urine test in the previous test.

### BRIEF DESCRIPTION OF THE DRAWINGS

FIG. 1a is an embodiment of one test strip of the present invention having two hCG urine tests of different sensitivities used for both the first and second test at the desired interval.

FIG. 1b and FIG. 1c are embodiments of the present invention showing alternative test strip variations within the scope of the present invention.

FIG. 2 is a flow chart of the method of the present invention.

FIG. 3 is a flow chart of an embodiment of the present invention method.

### DETAILED DESCRIPTION OF THE INVENTION

The present invention takes advantage of the fact that a viable ongoing or progressing pregnancy has a predictable and expected rise in hCG concentration in blood and urine during the initial 8 to 10 weeks of the first trimester. By using different sensitivities of home test strips over a desired or selected period of time it can be established that the hCG levels in the urine are increasing and thus the pregnancy is progressing and viable.

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “home pregnancy test kit” refers to a test kit that involves a patient being able to do the test at home, for example, a urine test which indicates a positive or negative result by a color change or other means such as a digital output. The home test is designed to be used by someone with no medical experience and as such the urine type tests are ideal. The home test kits are sensitive to the presence of hCG in urine and change color, or otherwise indicate, when above the threshold sensitivity to the hCG is detected in the particular test. In general, single sensitivity hCG testing is known and currently these products are marketed as a one test, either yes or no test, with a wide availability of sensitivities available from about 20 mIU/mL to well over 100 mIU/mL but have the problems associated with them as described above when used as a single test. One could easily make even less sensitive tests available with the current technology but until now it has been thought that only more sensitive tests were needed.

The present invention home pregnancy test kit and method allows one to test for pregnancy and then confirm that the pregnancy is active, ongoing and progressing. The prior art use of home pregnancy tests do not allow that to occur. By the nature of the serial test strip product and method, one can test and know that a woman is above one threshold and below the other in the test strip and on subsequent testing is above both thresholds indicating increasing hCG and thus an active viable pregnancy. The first test strip then would be the most sensitive for example at 20 mIU/mL. This would establish a potential pregnancy (i.e. conception) just as using the test strip by itself would do. However, at a desired interval from the first test strip, a second different test strip is used. The time in one embodiment for example would be 2, 4 or 6 days. In another embodiment 2 days between each test. One would also be concerned with when the first test is started and measurement from ovulation date would further add to the accuracy of the test, although, not absolutely necessary within the scope of the present invention.

The second or subsequent test strips will have at least one or more hCG tests wherein at least one test is less sensitive than the first test on the second or subsequent strip. Subsequent strips will be identical to the original second strip in many embodiments. In one embodiment, one test is the same as the first test strip and the second test less sensitive while in another embodiment the 2 tests are progressively less sensitive. For example, where the initial test strip had a 20 mIU/mL sensitivity, the second test strip with two tests would have a first test with about a 40 mIU/mL sensitivity and a second test with a 150 mIU/mL sensitivity. One could envision 3 or more tests on the second test strip, for example, the initial sensitivity and 3 additional tests of 40, 150 and 1000 mIU/mL respectively on one test strip giving 4 different sensitivity results.

For each additional test on the second test strip it would be desirable to have multiple second strips for continuing to take the test at appropriate intervals. For example, where there are 3 tests on the second test strip, each less sensitive than the initial test strip, one might include 3 of the second test strips to test for increasing amounts of hCG. Likewise, for ease of use, the first test strip could have the other less sensitive tests on it as well, thus, a manufacturer could produce one kind of strip for the first and second strips and the user would not be confused which one to use to begin. In this scenario, the ideal test would show one positive test on the first test and each successive test would show one more test positive in addition to the previously positive tests, indicating the progression of the pregnancy.

As used herein a “test strip” is a strip of the kind used for the purpose of placing urine on a particular spot which initiates an hCG urine color or other indicator test. In newer type testing the test strip could also be a digital type where an indicator screen displays a message such as pregnant or not pregnant instead of a simple color change. Typically, these known tests, test for the beta subunit of hCG, but other urine type tests could also be used where they meet the criteria for home urine testing. The strips of the present invention are frequently referred to as sticks but strips and other types of devices and names are well known. These types of test strips are well known in the art and for purposes of simplicity are all referred to as test strips.

While “strip” in one embodiment means a single device, for purposes of this invention, other embodiments covered by the term test strip includes two or more devices attached to one another for ease in putting urine on both at the same time. In yet another embodiment it refers to two or more separate strips designed to be used at the same time to get the results of the more and less sensitive tests at the same time. Other embodiments where there are more or less strips can be envisioned, but the provision of tests of decreasing sensitivity as the pregnancy progresses is the concept of the present invention.

The current home tests available on the market are sensitive between about 20 mIU/mL to about 100 mIU/mL. It can be seen that one could pick one of these at the upper end of the range and lower end of the range and place both on the test strips for use in a first and second test. It is obvious to one of ordinary skill in view of this disclosure that if urine tests of greater or lower sensitivity are developed that these could also be utilized in the practice of the present invention. In one embodiment the difference in sensitivity of the two tests is at least 20 mIU/mL., in one other embodiment, the sensitivity of one test is between about 20 and 30 mIU/mL while the other test is a sensitivity above about 30 mIU/mL. It is well known that on average singleton hCG values during pregnancy run at about 100, 13 days after ovulation (gestational age of about 4 weeks) and around 1500, 20 days after ovulation with, under normal conditions, an essentially straight line increase in hCG values between those dates. It is clear from this disclosure then that by timing of the multiple tests, one can not only get an indicator of conception, but test for the ongoing viability of the pregnancy with relative accuracy in a manner that has not previously been achievable at home.

While the minimum for the invention is to have two data points created by two tests, one initial test of one sensitivity and a second test of lesser sensitivity, other embodiments of the invention can be envisioned such as to have 3 or more tests on each test strip to give even more clarity to the progress of the progressing pregnancy or to chart the progress over more than two test days. In the embodiment above with 3 tests, in addition to the initial hCG test, a test can be done on 3 determined subsequent days such that on the first day just the first test would be positive the second test day both the first and second test positive and so on for as many tests and test intervals as desired. Multiple tests would be handled in the same manner and one skilled in the art would be able to produce multiple tests in view of the present disclosure including the addition of more or less sensitive tests.

In general, the method of the present invention involves a first step of selecting a test strip according to the above testing description for a first test strip. If this first test is negative for the one or more tests on the first test strip, the individual is deemed not pregnant. If at least the most sensitive hCG test on the strip is positive then there is a first indicator of a pregnancy. In order to determine if it is an ongoing pregnancy, a second hCG home urine test is performed with at least one of the tests on the second test strip less sensitive than the first exam. If the interval between the tests is properly chosen (which can be done by one skilled in the art and given to the individual for home use as described above), then on the second test strip the less sensitive test will also be positive. One can see that it would be useful to have the second less sensitive test on the first strip so that if the less sensitive test is negative on the first test and then positive on the second test a greater confidence in the result can be had than one test on each strip.

The present method can be accomplished with just two tests at a single interval, but in one embodiment there are multiple urine tests at 3 or more times separated by a predetermined interval corresponding to the sensitivity of the selected hCG testing and the hCG urine concentration expected based on the date of the missed menstrual cycle or other determining factor. In one embodiment where serial tests of different sensitivity are used at each stage of testing the first test might be the most sensitive test perhaps 20 mIU/mL. If that test were positive and say 2 or 3 other less sensitive tests were negative, then a first test indicator would be passed. The second test would be taken at a time interval that would allow urine hCG to raise to the level of the sensitivity of the second test say for example 40 mIU/mL a couple days later. This scenario could be repeated over 3, 4 5 or more times at appropriate intervals till a pattern emerges. Therefore, in the embodiment with 4 tests, the first test would have one positive, second two positive, third three positives, and so on for an ongoing pregnancy. A failure for a successive test to show a positive would indicate (if the time period is correct) a pregnancy that had not advanced, for example, by miscarriage or ectopic pregnancy. The result is a picture of a pregnancy where the hCG levels are increasing using a readily available home based hCG urine test.

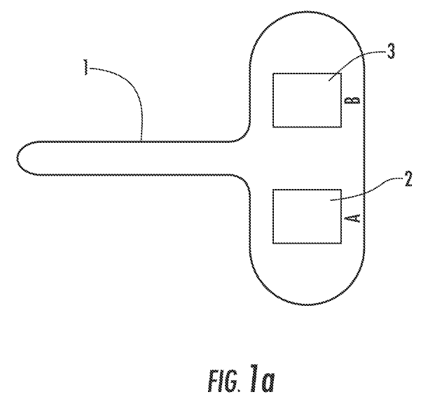

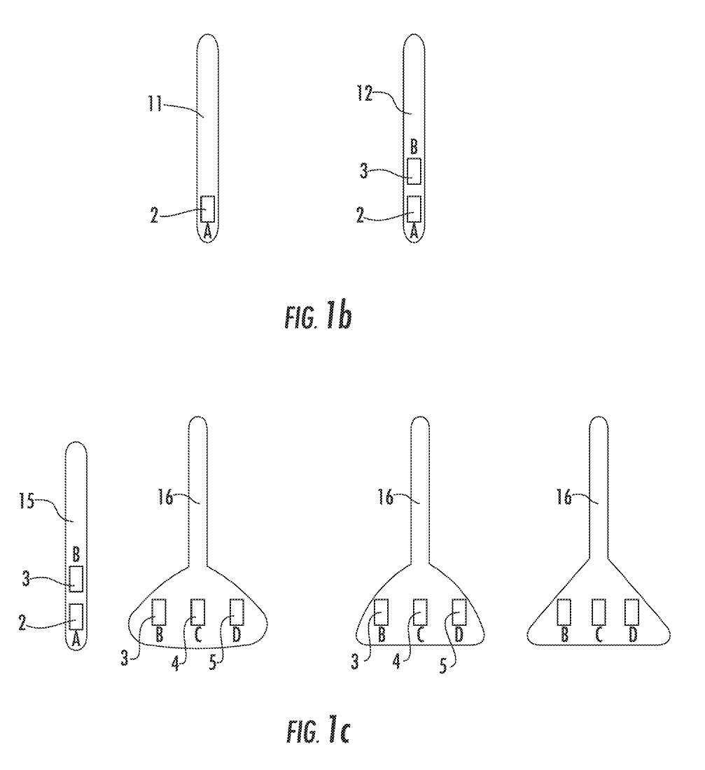

Now referring to the figures FIG. 1a is a single embodiment of one test strip of the kit of the present invention. The test strip 1 comprises a first urine test 2 and a second urine test 3. The two tests have a different sensitivity to the presence of hCG. By placing a urine sample over both test 2 and 3 two different tests results occur at once. In the first test the test “a” would be positive and second less sensitive test “b” negative. In a subsequent test at the appropriate interval test b would be positive indicating an ongoing pregnancy. While FIG. 1 indicates a two stage test with the same type of device used for both tests as described above the number of tests on each test can vary within the parameters of the working of the present invention and tests can be on multiple tests strips as noted above.

FIG. 1b shows a kit of the present invention with a first test strip having the initial test 2 and a second test strip 12 having the initial test 2 and a second test 3 of lower sensitivity. FIG. 1c shows yet another embodiment with first test strip 15 having a first 2 and second 3 hCG test of differing sensitivity. Also shown are three secondary tests strips 16 with tests 3, 4 and 5 each of decreasing sensitivity. Using the first test strip would indicate if test 2 is positive a potential pregnancy and each of the next strips would show advancing pregnancy as taught above and in the example. In this embodiment there are 4 test strips where as each test is taken the next test would show a positive result if the tests are used at the proper intervals.

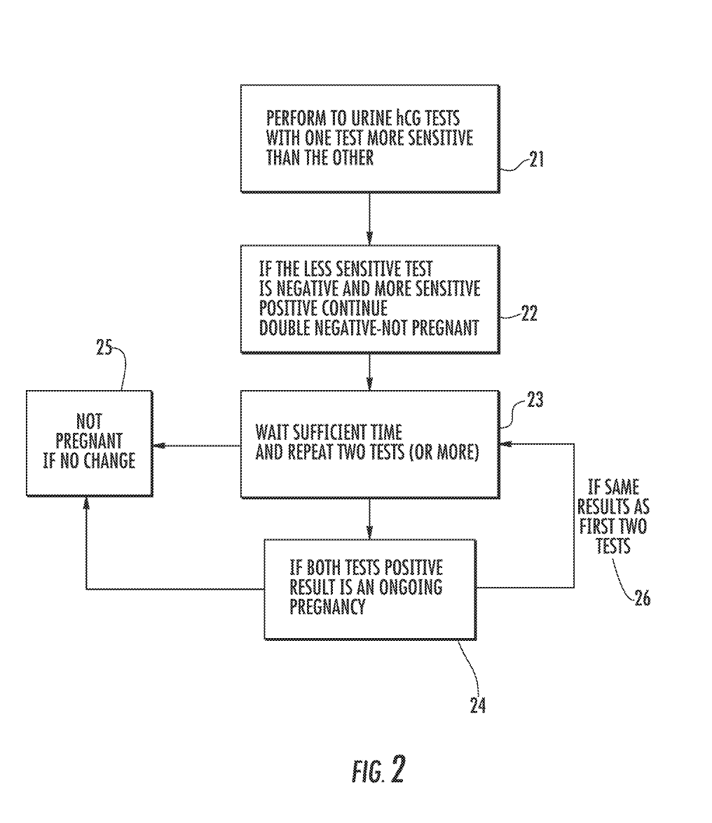

FIG. 2 is a flow chart of an embodiment of the process of the present invention. Initially a person deciding they wish to see if they have an ongoing progressing pregnancy would perform a urine test with a first test strip, the test strip having 2 urine hCG tests on it with one test more sensitive than the other 21. If the more sensitive test is positive and the less sensitive test negative then the process can continue 22. If both tests are negative the person is not pregnant within the sensitivity of the test. If both are positive the pregnancy is beyond the sensitivity of the test. One way of avoiding this problem is to have a wide difference between the test sensitivities or to have several tests each with different sensitivities on each test strip. One could in embodiments of this type have 3, 4, 5, 6 or more tests of differing sensitivities. In that case some positive and some negative would indicate movement to the next step.

After waiting a sufficient predetermined time for the patient's hCG levels to increase the two (or more) tests can be repeated 23. If on second repeat the second test is positive or in the case of multiple tests more tests are positive than the previous test strip test then the test is positive for an ongoing pregnancy with increasing hCG levels.

If the test is the same after the period of time 26 the test can be repeated after sufficient time once again. In that case one can assume that the person is not pregnant 9 or that the pregnancy is not advancing 25. If the both tests on the second test strip are positive the result is an ongoing pregnancy 24. One skilled in the art can clearly increase the number of tests strips sensitivities and the like within the scope of the claimed invention. Nothing in the claims or these examples are intended to limit the invention.

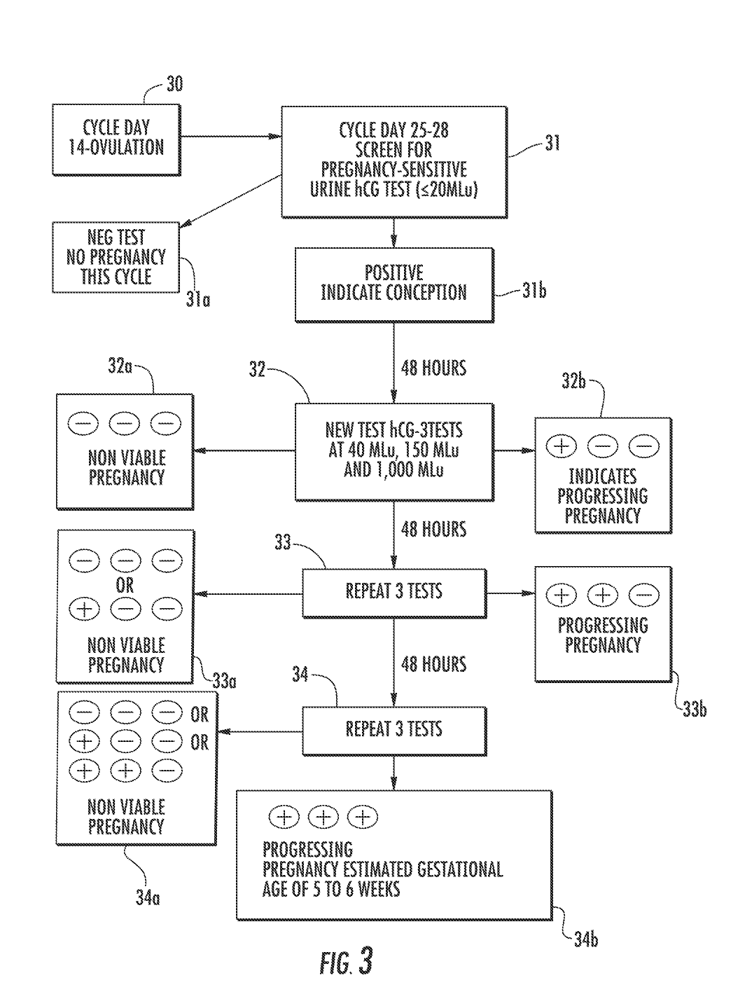

FIG. 3 is a flow chart of a specific embodiment of the present invention. If the patient has a 28 day cycle, there is an ovulation around day 14 of the cycle. In this particular embodiment a first hCG test is taken at around cycle day 25 to 28 31 using a most sensitive test at about 20 mIU/mL. This is the standard most sensitive test available on the market. This test if negative 31a indicates no pregnancy and if positive 31b indicates conception had occurred (but not an indication of a viable ongoing pregnancy).

If there is conception 31b then after a period of 48 hours, a new 3 less sensitive tests 32 urine test is administered. In this embodiment the test has 3 tests at 40, 150 and 1000 mIU/mL respectively. If all three tests are negative (indicated by minus sign) then the indication is a non viable pregnancy 32a. If the first test is positive (indicated by the first plus sign) and the other two tests negative, this indicates a progressing pregnancy 32b. If the tests indicate viable pregnancy 32b then a repeat of three tests is done after another 48 hours 33. If all the tests are negative or only the first test is positive that is now an indication of non viable pregnancy 33a. If the first two tests are positive this is an indication of continued ongoing viable pregnancy 33b. If a progressing pregnancy 33b is indicated the 3 tests are repeated a third time 34. Anything other than all three tests being positive would indicate a non viable pregnancy 34a but all three tests positive at this point would indicate progressing pregnancy with an estimated gestational age of about 5 to 6 weeks 34a.

### EXAMPLE

On the first day that the menstrual period of an individual is noted to be missed, the individual is instructed to use the first hCG urine test, which is a sensitive 20 mIU/mL test, to screen for pregnancy. The kit may contain more than one first screening test, as establishing this early baseline is critical, thus allowing testing over several days if needed. Once pregnancy is established with the first test, the individual now begins testing with the serial (second) indicator strips. At this early gestation, the hCG level should have one doubling cycle every 2 days. Two days after the initial test becomes positive, the individual is instructed to use the first serial test. If the pregnancy is progressing, the first will now be positive, given that the sensitivity of this second test approximates a 40 mIU/mL threshold. In a test with 2 addition tests of decreasing sensitivity the second and third windows will still be negative. The next test will be taken 4 days (two doubling cycles) after the previous test. At this point the first and second windows will indicate a positive result, with a sensitivity in the second window approximating a 150 mIU/mL threshold, and the third window still being negative. The final test is used 6 days later. All windows will be positive at this point if the hCG level has reached the 1000 mIU/mL threshold. The time frame of this testing protocol covers what would correlate to an estimated gestational age between 4 and 6 weeks. This is a critical time as beyond this point, most pregnancies can now be identified on transvaginal ultrasound. Beyond 6 weeks of gestation, ultrasound demonstration of fetal cardiac activity is the gold standard for confirming pregnancy viability. If at any point in the testing any of the successive tests are negative, the individual will contact the physician to determine the problem with the pregnancy, or lack thereof, indicated by the progressive negative finding with the test.

What is claimed is:

1. A home pregnancy test kit comprising two or more test strips, a first test strip comprising a first hCG home urine test of a first hCG sensitivity and the second and subsequent tests strips comprising at least one hCG home urine test less sensitivity to hCG than the first hCG home urine test.

2. A kit according to claim 1 wherein the first test strip has a hCG test with a hCG sensitivity of between about 20 and 30 mIU/mL and the second and subsequent test strips have one or more tests having a hCG sensitivity from about greater than about 30 mIU/mL

3. A kit according to claim 1 wherein the sensitivity of the first test one the first test strip is about 20 mIU/mL and the second and subsequent test strips have 3 tests having an hCG sensitivity of about 40 mIU/mL, 150 mIU/mL and 1000 mIU/mL respectively.

4. A kit according to claim 1 wherein the second and subsequent tests strips each comprises two or more hCG tests of decreasing sensitivity.

5. A kit according to claim 4 wherein at least two of the hCG tests of decreasing sensitivity are less than the first sensitivity.

6. A kit according to claim 4 wherein at least one of the two or more hCG tests has a sensitivity about equal to the first sensitivity.

7. A kit according to claim 1 wherein all the test strips are identical.

8. A kit according to claim 1 wherein the first test strip further comprises 1 or more additional hCG tests of a sensitivity identical to at least one of the hCG tests on the second and subsequent test strips that is less sensitive that the first sensitivity.

9. A method of confirming an ongoing pregnancy in an individual comprising the steps of:
a) performing on the individual, a first hCG urine test of a first sensitivity with a first test strip at a given time;
b) performing on the individual at a predetermined time after the first test, a second urine hCG test with a second test strip, the second test strip having at least one hCG urine test having a lesser sensitivity to hCG than the first sensitivity; and
c) determining an ongoing pregnancy confirmation when the first test on the first test strip is positive and the less sensitive test on the second strip is positive.

10. A method according to claim 9 wherein there are 2 or more tests on each second test strip each test having a different sensitivity.

11. A method according to claim 9 wherein the first test strip has 2 or more tests on it at least one of the additional tests being identical to at least one test on the second test strip which is less sensitive that the first sensitivity.

12. A method according to claim 11 where confirmation is determined when the less sensitive test on the first strip in the first urine test is negative and positive in the second urine hCG test.

13. A method according to claim 10 wherein one of the 2 or more tests has a test sensitivity about equal to the first sensitivity and the remaining tests have a sensitivity progressively less that the first sensitivity.

14. A method according to claim 10 wherein the 2 or more tests are each successively less sensitive to hCG than the first sensitivity.

15. A method according to claim 10 where in the difference in sensitivity between the first test and one less sensitive test is at least 20 mIU/mL.

16. A method according to claim 9 wherein the first sensitivity is about 20 mIU/mL.

17. A method according to claim 9 wherein the given time is determined based on the sensitivity of the tests and the anticipated progress of hCG urine concentration in a normal ongoing pregnancy.

18. A method according to claim 9 wherein the number of second and subsequent tests is equal to the number of hCG tests on the second test strip less sensitive to hCG than the first sensitivity.

19. A method of testing in an individual, the progress of a pregnancy comprising testing the individual at, at least 2 predetermined time intervals with a hCG urine test the sensitivity of at least one hCG urine test taken at each interval is successively less sensitive than at least one hCG urine test in the previous test.

20. A method according to claim 19 wherein there are the same two or more tests taken at each predetermined time.
